(19) United States
(12) Patent Application Publication
(10) Pub. No.: US 2020/0061223 A1
(43) Pub. Date: Feb. 27, 2020

(54) DISINFECTION SYSTEM

(71) Applicant: GENTEX CORPORATION, ZEELAND, MI (US)

(72) Inventor: Jason D. Hallack, Holland, MI (US)

(73) Assignee: GENTEX CORPORATION, ZEELAND, MI (US)

(21) Appl. No.: 16/533,919

(22) Filed: Aug. 7, 2019

Related U.S. Application Data
(60) Provisional application No. 62/720,226, filed on Aug. 21, 2018.

(51) Int. Cl. A61L 2/10; B60S 1/64; B60N 2/00; A61L 2/28

(52) U.S. Cl. CPC A61L 2/10; B60S 1/64; B60N 2/002; A61L 2202/14; A61L 2202/11; A61L 2202/25; A61L 2/28

(57) ABSTRACT

A disinfection system is disclosed. The disinfection system comprises a light source, a first sensor, and a controller. The light source is configured to emit germicidal light in a vehicle interior. The first sensor is operable to detect the presence of at least one of bacteria, viruses, dirt, and grime. The controller is configured to selectively at least one of activate, project, and steer light emissions from the light source based at least in part on sensor data. Optionally, vehicle interior disinfection system may further comprise a second sensor. The second sensor may be operable to capture occupant data. Further, the controller may be further operable to determine if the vehicle interior is occupied.

### CROSS-REFERENCE TO RELATED APPLICATION

This application claims priority under 35 U.S.C. § 119(e) to U.S. provisional Application No. 62/720,226 filed on Aug. 21, 2018, entitled “VEHICLE DISINFECTION SYSTEM,” the disclosure of which is hereby incorporated by reference in its entirety.

### FIELD OF THE INVENTION

The present invention generally relates to an illuminated disinfection system for a vehicle interior and, more particularly, to light assemblies configured to disinfect a vehicle interior.

### BACKGROUND OF THE INVENTION

In occupied spaces, such as vehicle passenger compartments and hotel rooms, cleanliness is important. In the setting of vehicle passenger compartments, especially those in shared vehicles or in the on-demand type, cleaning and disinfecting is desired between users. Although cleanliness is preferred between users, vehicles in many car rental and shared vehicle programs are not professionally or adequately cleaned and disinfected between users. In the setting of hotel rooms, cleaning may not be thorough and complete enough—especially considering the increased likelihood of bacteria, viruses, dirt, and/or grime being present given the nature of hotels. This inadequacy leads to customer dissatisfaction and improved risk of spreading disease and other conditions that can be easily prevented through improved cleaning. Accordingly, methods or systems that can quickly and effectively clean on occupiable space, such as a vehicle interior or a hotel room between users would improve the hygiene, customer satisfaction, and efficiency at which different users can share a vehicle.

Further, disinfection systems often use compounds and lights, such as ultra-violent light, which can be harmful to occupants exposed and damaging to materials in the vehicle interior. Accordingly, methods or systems that can reduce, minimize, or eliminate harmful exposures to occupants and materials in the vehicle interior are needed.

### SUMMARY OF THE DISCLOSURE

In accordance with the present disclosure, the disadvantages and problems associated with protecting an electro-optic element at smaller spacing between the electro-optic element and the dust cover have been substantially reduced or eliminated.

In accordance with one embodiment of the present disclosure, a disinfection system is disclosed.

The disinfection system comprises a light source, a first sensor, and a controller. The light source is configured to emit germicidal light in a vehicle interior. The first sensor is operable to detect the presence of at least one of bacteria, viruses, dirt, and grime. The controller is configured to selectively at least one of activate, project, and steer light emissions from the light source based at least in part on sensor data. Optionally, vehicle interior disinfection system may further comprise a second sensor. The second sensor may be operable to capture occupant data. Further, the controller may be further operable to determine if the vehicle interior is occupied.

In accordance with another aspect of the present disclosure, a method for disinfecting a space is disclosed. The method for disinfecting the space comprises sensing the presence of at least one of bacteria, viruses, dirt, and grime on a surface of the space and at least one of selectively activating, projecting, and steering a germicidal light emission from a light source, based at least in part on the sensed presence of at least one of bacteria, viruses, dirt, and grime.

The technical advantages of certain embodiments of the present disclosure include reducing the risks of spreading diseases and other conditions and reducing harmful exposures of UV light and chemical compounds to both occupants and materials. The present disclosure enables cleaning and disinfection between different users of a space, thereby reducing the risks of spreading diseases and other conditions by not only providing a mechanism for cleaning and disinfection, but by also enabling automatic initiation upon detecting an occupant and the occupant's subsequent absence. Further, the present disclosure has reduces harmful exposures of UV light and chemical compounds to occupants by operating disinfection cycles during their absence and stopping the cycles when an occupancy interrupts the cycle. Additionally, the damaging exposures of UV light to materials are reduced by enabling the targeting of contaminated or unclean areas, thereby reducing the exposure to clean areas.

These and other aspects, objects, and features of the present disclosure will be understood and appreciated by those skilled in the art upon studying the following specification, claims, and appended drawings. It will also be understood that features of each example disclosed herein may be used in conjunction with, or as a replacement for, features of the other examples.

### BRIEF DESCRIPTION OF THE DRAWINGS

The following is a description of the figures and the accompanying drawings. The figures are not necessarily to scale, and certain features and certain views of the figures may be shown exaggerated in scale or in schematic in the interest of clarity.

In the drawings:

FIG. 1 is a cockpit view of a vehicle including a rearview mirror assembly, according to some aspects of the present disclosure;

FIG. 2 is a front isometric view of a rearview mirror assembly for a vehicle, according to some aspects of the present disclosure;

FIG. 3 is a partially exploded view of the rearview mirror assembly of FIG. 2 according to some aspects of the present disclosure;

FIG. 4 is a cross-sectional view taken along the line of FIG. 1, further illustrating the rearview mirror assembly according to some aspects of the present disclosure;

FIG. 5 is a schematic flow diagram of a disinfection system according to some aspects of the present disclosure;

FIG. 6 is a isometric view of a vehicle compartment, according to some aspects of the present disclosure; and

FIG. 7 is a flow chart demonstrating a disinfection routine for a disinfection system for a vehicle interior, according to some aspects of the present disclosure.

### DETAILED DESCRIPTION OF THE PREFERRED EMBODIMENTS

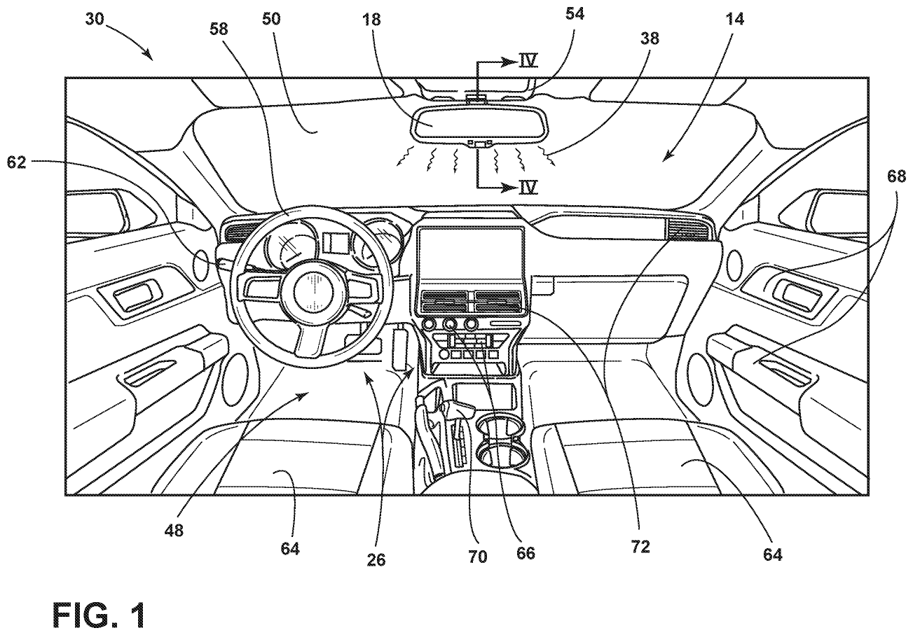

For purposes of description herein the terms “upper,” “lower,” “right,” “left,” “rear,” “front,” “vertical,” “horizontal,” and derivatives thereof shall relate to the device as oriented in FIG. 1. However, it is to be understood that the device may assume various alternative orientations and step sequences, except where expressly specified to the contrary. It is also to be understood that the specific devices and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

As used herein, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

In this document, relational terms, such as first and second, top and bottom, and the like, are used solely to distinguish one entity or action from another entity or action, without necessarily requiring or implying an actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

Referring now to FIGS. 1-5, the reference numeral 10 generally designates a disinfection system. Disinfection system 10 may be designed for any space. The space may be an occupiable space such as, but not limited to, a vehicle interior 14 or a room, such as a hospital or a hotel room. A vehicle interior 14 is depicted for exemplary purposes only. Disinfection system 10 may include a rearview mirror assembly 18. The rearview mirror assembly 18 includes a light assembly 22 configured to selectively illuminate a control region 26 in a vehicle 30. The rearview mirror assembly 18 additionally includes at least one light source 34 positioned within the light assembly 22 and configured to emit light 38. The rearview mirror assembly 18 additionally includes at least one sensor 42 configured to scan the control region 26 in the vehicle 30 to capture occupant data. The rearview mirror assembly 18 also includes a controller 46 configured to process the occupant data to determine if the control region 26 is occupied or unoccupied in addition to controlling the light assembly 22 to activate at least one of the light sources 34 to emit light 38 on the control region 26 of the vehicle 30.

Referring now to FIG. 1, a cockpit view of the vehicle 30 including the rearview mirror assembly 18, according to some aspects of the present disclosure, is provided. As illustrated, the rearview mirror assembly 18 is mounted to a front windshield 50 in the vehicle interior 14 where the mirror assembly 18 may project light 38 to illuminate, sense, and/or clean a passenger compartment 48. In some aspects, the rearview mirror assembly 18 may be mounted to a vehicle header 54 of the vehicle 30. The vehicle interior 14 additionally includes the control region 26 including many of the controls a driver or passenger may interact with and touch when using or riding in the vehicle 30. For example, in some aspects the control region 26 may include a steering wheel 58, a blinker stalk 62, a dash panel, radio and climate controls 66, door handles 68, a transmission shifter 70 and/or vent duct openings 72. When the passenger compartment 48 is unoccupied, the rearview mirror assembly 18 may project light 38 used to disinfect or clean the control region 26, seatbelts, vehicle seats 64, and/or passenger compartment 48.

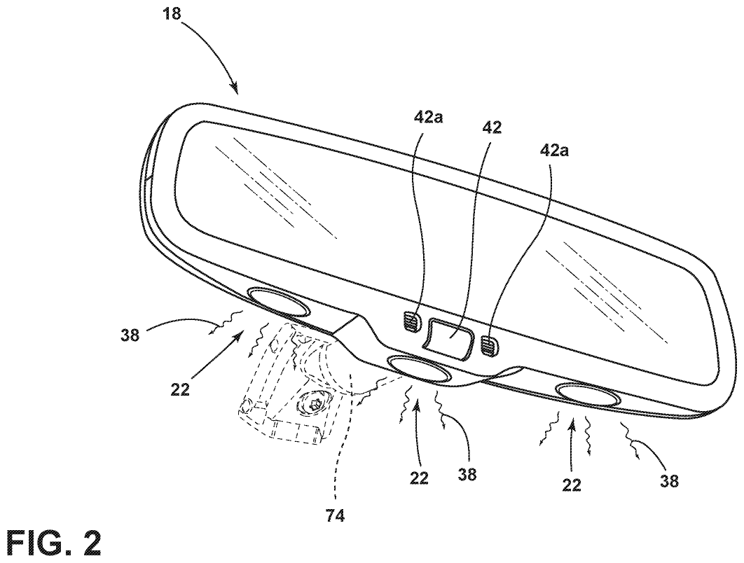

Referring now to FIG. 2, the illustrated rearview mirror assembly 18 can be an interior rearview assembly positioned within the vehicle interior 14 (see FIG. 1). When the rearview mirror assembly 18 is an interior rearview assembly, the rearview mirror assembly 18 may be connected to a mount 74, which is adapted to be mounted inside the vehicle 30 in a location proximate to or on the windshield 50 (see FIG. 1). The rearview mirror assembly 18 may include one or more light assemblies 22 used to generate and project light 38 or electromagnetic radiation into the vehicle interior 14. In some aspects, one or more sensors 42 and 42a may be used to detect a variety of different conditions and/or parameters in the vehicle interior 14 (e.g., occupancy, cleanliness, bacteria, viruses, molds, and/or fungi). It should be noted that the present disclosure is equally applicable to exterior mirrors, as well as other optical assemblies, including, for example, switchable eyewear, mirror assemblies, architectural window assemblies, aircraft window assemblies, filter assemblies, and vehicle windows including other optical assemblies positioned within bezels and housings.

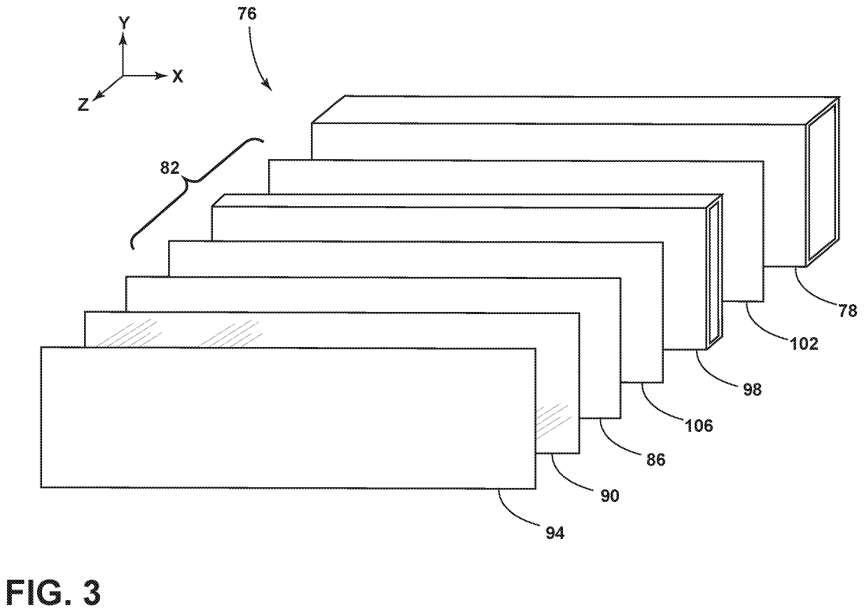

Referring now to FIG. 3, the rearview mirror assembly 18 may include an electro-optic device 76 having, in order from a rear position (e.g., vehicle forward) to a front position (e.g., vehicle rearward), a mirror light source 78, a display 82, an optical substrate 86, a reflective polarizer 90 and an electro-optic element 94. It will be understood that the reflective polarizer 90 may be positioned on either a vehicle-forward or vehicle-rearward surface of the optional substrate 86 without departing from the teachings provided herein. The mirror light source 78, or light engine, is configured to backlight the display 82 by providing light to a rear of the display 82. Light generated by the mirror light source 78 may be polarized or unpolarized. Light from the mirror light source 78 moves in a Z-direction through the rearview mirror assembly 18, through the display 82, and toward the electro-optic element 94. In the depicted example, the display 82 is a liquid crystal display incorporating a liquid crystal medium 98 disposed between two polarizers, an entrance polarizer 102 and an exit polarizer 106. The mirror light source 78 and display 82 may extend the entire length of the rearview mirror assembly 18 creating a “full-display” assembly, or may only extend a portion of the length. It will be appreciated, however, that a concept of a “full-display” assembly, where the display 82 or a plurality of displays, located behind the electro-optic element 94, overlaps in projection onto a viewable surface of the rearview mirror assembly 18, with most or all of this viewable surface, is also contemplated by the various examples of the disclosure. The display 82, and/or mirror light source 78, may be angled (e.g., about 3° to about) 5° relative to the reflective polarizer 90 and optionally include an optical bonding adhesive disposed between the display 82, the reflective polarizer 90, and the optional substrate 86 and other locations.

The entrance and/or exit polarizers 102, 106 may include a reflective polarizer which may be a linear polarizer, an elliptical polarizer, or a circular polarizer and might include an optical retarder such as a quarter-wave plate or a half-wave plate. A wire-grid polarizer provides one example of a reflective polarizer that may be used for the entrance and/or exit polarizers 102, 106. Alternatively, a reflective polarizer may include a polymer-based film structure including at least one optically anisotropic layer. Such polymer-based film structure is generally referred to herein as an anisotropic polymer-based film (APBF). Non-limiting examples of APBF's are provided by a multilayered polymer film, including a body of alternating layers of crystalline-based polymer and another selected polymer, or by micro-structured film-based polarizers, such as brightness enhancement films, or by dual brightness enhancement films.

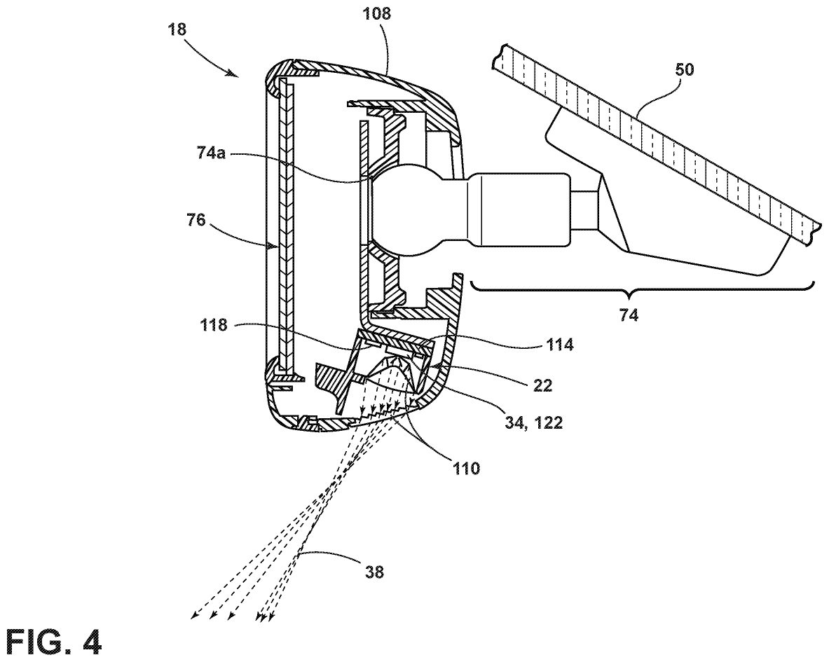

Referring now to FIG. 4, a cross-sectional view taken along the line of the rearview mirror assembly 18 illustrated in FIG. 1 is provided. The rearview mirror assembly 18 includes the light assembly 22 including components that use refraction, reflection, scattering, absorption, interference, and diffraction to alter the shape, pattern, intensity, distribution, spectral distribution, orientation, divergence, and other properties of the light emitted by the light source 34. The electro-optic device 76 may be positioned in a housing 108 where the housing 108 encloses a mounting bracket 74a operatively coupled to the mount 74 positioned on the windshield 50. More specifically, the light assembly 22 may include secondary optics 110 that may include an inner lens consisting of refracting and total internal reflection (TIR) reflecting optics and/or secondary optics 110 including a lens using refracting principals. A substrate 114 may include a circuit device 118 attached to at least one of its main surfaces. Circuit devices 118 may include integrated circuits (ICs) and/or discreet circuit devices, such as resistors, transistors, capacitors, inductors, and diodes. As illustrated, substrate 114 is a printed circuit board (PCB) configured to electrically couple the various circuit components and elements located on its main surface to each other. Substrate 114 also includes an electrical connector (not shown) coupled to the circuit devices on the surface of the substrate 114, and configured to provide power and electrical signals to those devices. In some aspects, the light source 34 positioned on the substrate 114 may include a LED 122 used to generate light 38 and/or electromagnetic radiation into the vehicle interior 14 and passenger compartment 48.

In some aspects, the substrate 114 includes at least one light source 34 or LED 122 that is electrically and physically connected to the substrate 114 using conventional soldering techniques. In some aspects, the light source 34 may include a single light LED device that includes one or more semiconductor LED chips and/or phosphors configured to complement one another to form white light. In other aspects, the at least one light source 34 includes a UV LED device that includes one or more semiconductor LED chips and/or phosphors configured to complement one another to form UV light. In some aspects, the LEDs can project a wavelength from about 100 to about 900 nanometers, from about 100 to about 700 nanometers, or from about 100 to about 400 nanometers. In some aspects, the at least one light source 34 may include one or more UV LEDs positioned to project visible light (400 nanometer to 700 nanometer), UV-A light (315 nanometer to 400 nanometer), UV-B light (180 nanometer to 315 nanometer), UV-C light (100 nanometer to 180 nanometer), or a combination thereof into the passenger compartment 48. In some aspects, the at least one light source 34 includes a first light source configured to emit UV-C light into the vehicle interior 14 or control region 26. In other aspects, the at least one light source 34 further includes a second light source configured to emit white light into the vehicle interior 14 or control region 26.

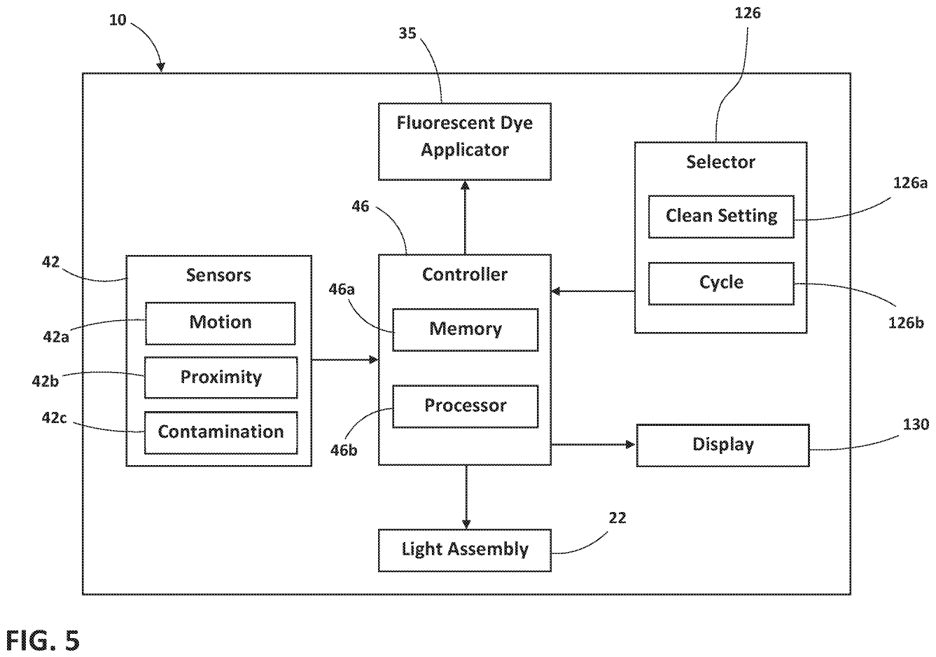

Referring now to FIG. 5, a schematic diagram for a disinfection system 10. Disinfection system 10 may be disposed in any space, such as an occupiable space. For example, the space disinfection system 10 may be a vehicle passenger compartment or a hotel room. Further, disinfection system 10 may be disposed within a device of the space such as a fire detector system or a rear-view mirror. Disinfection system 10 comprises, a controller 46 in communication with one or more of, one or more selector 126, one or more sensor 42, one or more light assembly 22, one or more fluorescent dye applicator 35, and one or more visual display 130.

Controller 46 comprises a memory 46a which stores instructions executable by a processor 46b. Controller 46 additionally receives input information from a selector 126 where the selector 126 can include, for example, the desired clean setting 126a and/or type of cleaning setting 126b. Upon selecting the desired parameters to be factored into the performance of the disinfection cycle, the selector 126 sends the input information concerning the disinfection cycle to the controller 46 where the processor 46b controls the at least one light assembly 22 to project the desired light 38 into the space. Controller 46 additionally may receive input/information related to one or more environmental condition within the space.

Sensor 42 is a device configured to detect a desired parameter of the environmental condition in the space. The environmental condition may comprise the occupancy and/or cleanliness of the space. Further, sensor 42 may relay the corresponding input information concerning a respective environmental condition to controller 46. Sensor 42 may be, but is not limited to, a motion sensor/detector 42a, a proximity sensor/detector 42b, infrared sensor, a contamination or cleanliness sensor/detector 42c, or any other sensor suitable to perform the operations described herein.

As provided by various aspects of the present disclosure, at least one sensor 42 may be configured to scan the space to capture occupant data. For example, the at least one sensor 42 may include an imager, a bed sensor, a seat sensor, a seatbelt sensor, a door opener event sensor, a motion sensor, a proximity sensor, or a combination thereof. Further, sensor 42 may be configured to emit light to aid in sensing.

Additionally, at least one sensor 42 may be configured as a contamination or cleanliness sensor/detector 42c. Contamination or cleanliness sensor/detector 42c may be any device operable to detect the presence of bacteria, viruses, dirt, and/or grime. For example, contamination or cleanliness sensor/detector 42c may be optical and/or mechanical based. Optical based sensors may function by measuring a change in fluorescence, absorbance, and or luminescence of a surface. In fluorescence based optical sensors, the surface is sprayed with a fluorescent dye. Mechanical based sensors may be based on quartz crystal microbalance or cantilever technology. Quartz crystal microbalance detect resonance frequency change that results from increased mass on a sensor surface due to analyte binding. Cantilever sensors typically comprise a bioreceptor-functionalized microcantilever which oscillates at a particular resonant frequency. The resonant frequency of the cantilever changes due to induced mechanical bending upon an increase in mass on the sensor surface.

Light assembly 22 comprises one or more light source 34. Light source 34 may be any device configured to emit visible and/or non-visible light. Further, light source 34 may be strobed at a controlled frequency. In some examples, the light source 34 may be configured to emit visible light, infrared light (e.g., near-infrared and/or far-infrared) and/or ultraviolet light. Visible light examples of light from the light sources 34 may have a color temperature of, from about 1700 k to about 2700k. The color temperature of the one or more light sources 34 may be variable across the color temperature range. Examples of the light sources 34 configured to emit infrared light, the infrared light may be used with one or more guidance systems (e.g., scanning and control systems) as described in greater detail. Examples of the light sources 34 which emit ultraviolet light, the ultraviolet light alone or in combination with other features (e.g., TiO₂ coatings, films and/or paints) may be configured to provide cleaning, sanitation, and/or sterilization of surfaces (e.g., control region 26, door handles 68, or other portions of vehicle 30/passenger compartment 48). For example, the ultraviolet light may be used in a photocatalytic process to kill bacteria, viruses, and/or to eliminate dirt and grime. Thus, light source 34 may be configured to emit wavelengths of germicidal light. The germicidal light may comprise wavelengths of light ranging from approximately 260 nanometers to about 270 nanometers. Such wavelengths may be emitted from one or more of the light sources 34, which may include mercury-based lamps, UV-LEDs (UV-C LED) lamps, and/or pulsed-xenon lamps.

Generally, light source 34 may be light-emitting diodes, incandescent bulbs, and/or other light emitting sources. Additionally, light source 34 may also be configured to emit light which excites a fluorescent dye. In some examples, the light may be referred to as an excitation emission. The excitation emission may be infrared, visible, and/or ultraviolet light. In such examples, a fluorescent dye may be applied by a fluorescent dye applicator 35 to space or a zone thereof. For example, the dye may be applied to control region 26 of passenger compartment 48. A fluorescent dye applicator may be any device operable to apply a fluorescent dye to a surface of passenger compartment 48. For example, fluorescent dye applicator may comprise a sprayer operable to deliver a mist of fluorescent dye onto the compartment surface. A fluorescent dye may be any solution operable to cause bacteria, viruses, dirt, and or grime to fluoresce when illuminated with an excitation emission, many of which are well know in the art.

Selector 126 is any device capable to input information. For example, selector 126 may be a dial, a button panel, and/or a touch screen. Selector 126 may include, for example, the desired clean setting 126a and/or type of cleaning setting 126b. Further, selector 126 is communicatively connected with controller 46 and is operable to send the input information to controller 46.

Display 130 is any device operable to display information to a user regarding disinfection system 10. Further, display 130 is communicatively connected to controller 46. For example, display 130 may be a personal computer such as a smart phone or a display fixed in a vehicle. Additionally, display 130 may be integrated with selector 126 such that display 130 is touch screen, enabling the input of information via the display screen.

In operation, selector 126 communicates input information to controller 46. According to the input information, controller 46 controls other parts of the disinfection system 10 to carry out a relevant mode of operation.

In some aspects of the present disclosure, sensor(s) 42 send corresponding input/information concerning respective environmental conditions to controller 46 where processor 46b derives setting corresponding to when, the type, the location, and/or the intensity of light 38 to be emitted for disinfection. Accordingly, when a disinfection cycle is to begin, controller 46 sends a command signal to light assembly 22 to emit light in accordance with the derived settings. Further, at the completion of the disinfection cycle or interruption due to a change in occupancy or input information, controller 46 may send a command signal to light assembly 22 to stop the disinfection cycle. Moreover, controller 46 may send appropriate information concerning a status of disinfection system 10 to visual display 130 where information may be visually presented to the user.

In other aspects, sensor 42 may be configured to scan the space and capture occupant data. The occupant data is communicated to controller 46. In response to the occupant data, controller 46 may process the occupant data and determine if the space is occupied or unoccupied and control the light assembly 22 to activate or deactivate at least one light source 34 to emit germicidal light in the space. For example, if an occupancy is detected during a disinfection cycle, the cycle may be stopped. Additionally, if an occupancy is detected, a disinfection cycle may be initiated upon cessation of an occupancy detection. This disinfection cycle may be directed to a space as a whole, a previously occupied portion thereof, an unoccupied portion thereof, and/or a control region 48.

Further, disinfection system 10 may perform occupancy detection by using the at least one sensor 42 including, for example, motion sensor 42a, proximity sensor 42b, contamination or cleanliness sensor 42c, image sensor, seat sensor, seatbelt sensor, or door opener event sensor. Additionally, disinfection system 10 may communicate or interface with a CAN and/or LIN vehicle bus to determine occupancy.

In some aspects, sensor 42 detects a contamination or cleanliness of passenger compartment 48. The detection may include illuminating a surface with light and imaging the surface to detect bacteria, viruses, dirt, and/or grime. Further, the detection may include fluorescent dye applicator 35 to pray the surface with a fluorescent dye before illumination. Detected contamination or cleanliness data may then be sent to controller 46 which in turn may initiate a disinfection cycle.

Upon initiation of a disinfection cycle, light source 34 may illuminate a region with detected bacteria, viruses, dirt, and/or grime with germicidal light. This illumination may be adjusted by controller 46 in accordance with the detected contamination or cleanliness data. For example, intensity may be increased when heightened levels of detect bacteria, viruses, dirt, and/or grime are detected. Further, the illumination may be adjusted to selectively target specific areas where bacteria, viruses, dirt, and/or grime are detected. For example, light assembly 22 may be configured to move, steer, activate, or otherwise influence the light to emit where the area to be targeted is positioned, such as a control region 26. In such embodiments, disinfection system 10 may comprise one or more positioning devices (e.g., a motor, actuator, etc.) which may correspond to electro-mechanical systems configured to adjust a position and/or projection direction of the one or more light sources 34. In static, or fixed, examples of the light sources 34, a light source 34 assigned to focus on various predefined points or areas of the passenger compartment 48 such as the control region 26 may be selected.

The present disclosure has the technical advantage of enabling vehicle cleaning and disinfection between different users to reduce the risks of spreading diseases and other conditions by not only providing a mechanism for cleaning and disinfecting the space by enabling automatic initiation upon detecting an occupant and the occupant's subsequent absence. Further, the present disclosure has the technical advantage of reducing harmful exposures of UV light and chemical compounds to occupants by operating disinfection cycles during their absence and stopping the cycles when an occupancy interrupts the cycle. Additionally, the present disclosure has the technical advantage of reducing the damaging exposures of UV light to vehicle interior materials by enabling the targeting of contaminated or unclean areas, thereby reducing the exposure to clean areas.

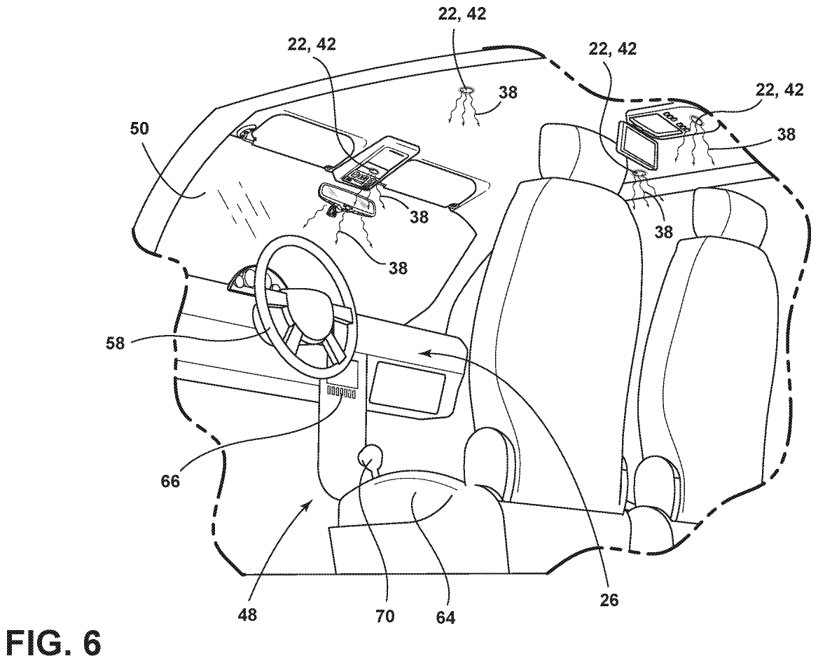

Referring now to FIG. 6, an isometric view of the vehicle interior 14 and passenger compartment 48 is illustrated. In some aspects, the number and positioning of the disinfection systems 10 is not meant to limiting. For example, as provided in FIG. 6, several disinfection systems 10 are positioned around the passenger compartment 48 to provide light 38 to various regions. In some aspects, the disinfection systems 10 may include the light assembly 22 and/or the sensor 42. In some aspects, the light assembly 22 and the sensor 42 may be positioned in various different positions with respect to each other in order to better clean and disinfect the passenger compartment 48. The disinfection system 10 positioned in the rearview mirror assembly 18 and/or roof control panel may be used to clean and disinfect the different portions of the control region 26 including, for example, the steering wheel 58, the front vehicle seats 64, the radio/climate controls 66, and the transmission shifter 70. The disinfection system 10 positioned in the passenger compartment 48 ceiling, roof, and/or back infotainment center may use the light assembly 22 and/or sensor 42 to clean and disinfect the different portions of rear area of the vehicle 30 including, but not limited to, for example, the back doors, the back seats, additional climate and/or entertainment controls. In some aspects, the disinfection system 10 is positioned in a rearview mirror assembly 18, a door, a console, and/or a ceiling panel. The number and positioning of disinfection systems including, for example, light assemblies 22, sensors 42, and mirror assemblies 18 may be varied and/or adjusted depending the on the desired application and cleanliness desired for the vehicle 30.

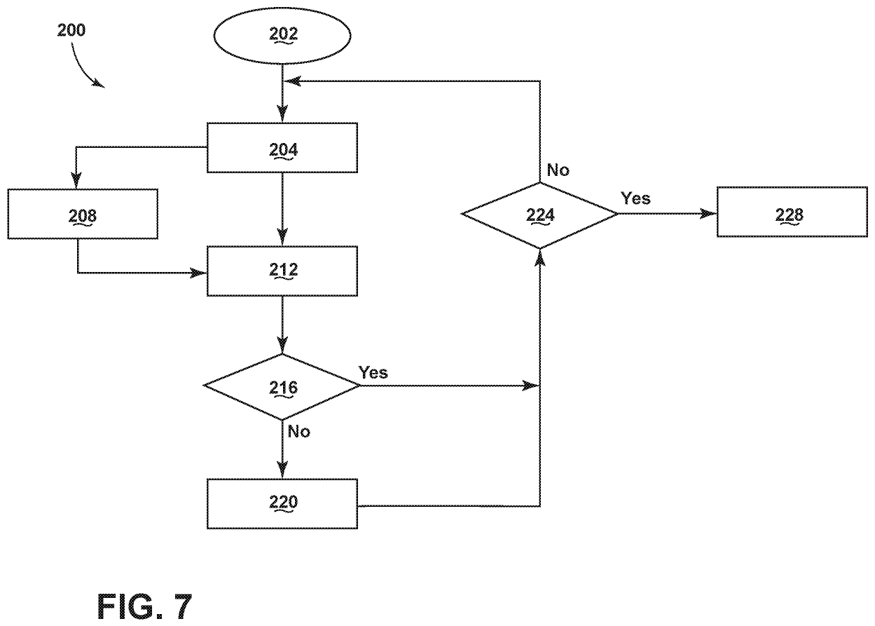

Referring now to FIG. 7, with continued reference to FIGS. 1-6, a method 200 for disinfecting a space is provided. The method 200 may begin with a step 202 that includes initiating the disinfection routine. In some aspects, the initiation of the disinfection routine may be triggered by sensor 42 detecting no passengers in the vehicle interior 14. In other aspects, the disinfection routine may be triggered by the user leaving the space as detected by sensor 42. In still other aspects, the disinfection routine may be triggered by a user programming a specific start time to begin the disinfection and/or cleaning.

Next is a step 204 of scanning the space using at least once sensor 42 configured to capture occupancy data. The one or more sensor 42 used to capture occupant data to determine if the space is occupied or unoccupied may include for example, but is not limited to, one or more motion sensors 42a, one or more proximity sensors 42b, one or more contamination sensors 42c, one or more IR body heat sensors and/or weight sensors positioned in vehicle seats 64, hospital or hotel beds and/or chairs.

Next is an optional step 208 of scanning the control region 26 for contamination including bacteria, viruses, and dirt and grime. In some aspects, one or more different types of contamination sensors 42c may be used to determine the presence of bacteria, viruses, mold, fungi, dirt, and grime. In some aspects, the disinfection routine may be initiated upon detection of contamination in the space. For example, the controller 46 of the disinfection system 10 may determine that the disinfection routine may not be required even if there are no occupants in the vehicle 30 if no contamination is detected.

Next is a step 212 of processing the occupant data and/or the contamination data to determine if control region 26 is occupied or unoccupied and/or if control region 26 is dirty or clean. Sensors 42 detect the one or more desired parameters of the environmental conditions in the space (steps 204 and 208) and send the corresponding input/information concerning the respective environmental conditions to the controller 46 where processor 46b derives the type and intensity of light 38 to be projected (see FIG. 5).

Next is a step 216 of determining if an occupant is detected. If no occupant is detected, a step of activating the ultraviolet radiation or illumination to clean and/or disinfect the vehicle interior 14, control region 26, and/or passenger compartment 48 begins in step 220. If yes, an occupant is detected, it is determined if the disinfection routine is complete in step 224. If yes, the routine is ended in step 228. If no, the method returns to step 204 where a next round of scanning the space for occupant data is initiated.

It will be understood by one having ordinary skill in the art that construction of the described device and other components may not be limited to any specific material. Other exemplary embodiments of the device disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the device as shown in the exemplary embodiments is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structure without departing from the concepts of the present invention, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above is merely for illustrative purposes and not intended to limit the scope of the device, which is defined by the following claims as interpreted according to the principles of patent law, including the Doctrine of Equivalents.

What is claimed is:

1. A disinfection system comprising:
a light source configured to emit germicidal light in a space;
a first sensor operable to:
detect the presence of at least one of bacteria, viruses, dirt, and grime; and
generate sensor data based on the detection; and
a controller configured to selectively at least one of activate, project, and steer light emissions from the light source based at least in part on sensor data.

2. The disinfection system of claim 1 further comprising a florescent dye applicator operable to apply a florescent dye to the space.

3. The disinfection system of claim 2 wherein the light source is further configured to emit an excitation emission.

4. The disinfection system of claim 1 wherein the light source is configured to emit UV light.

5. The disinfection system of claim 1 wherein the space is a vehicle interior.

6. The disinfection system of claim 5 wherein the light source is operable to emit onto a control region of the vehicle interior.

7. The disinfection system of claim 6 wherein the control region comprises at least one of a steering wheel, a turn signal stalk, a dash panel, at least one seat, and a seatbelt.

8. The disinfection system of claim 1 further comprising:
a second sensor, the second sensor configured to capture occupant data; and
wherein the controller is further configured to determine, based on the occupant data, whether the space is occupied.

9. The disinfection system of claim 8 wherein the second sensor is at least one of a motion sensor, a proximity sensor, a contamination sensor, a seat sensor, a seatbelt sensor, and a door opener event sensor.

10. The disinfection system of claim 8 wherein the controller is further configured to, based on the occupant data, stop a disinfection cycle upon determining the vehicle interior occupancy has changed.

11. The disinfection system of claim 5 wherein the disinfection system is disposed in a rearview mirror assembly.

12. The disinfection system of claim 1 wherein the space is a hotel room.

13. The disinfection system of claim 12 wherein the disinfection system is disposed in a fire detector system.

14. The disinfection system of claim 1 further comprising a visual display operable to visually present disinfection system information to a user.

15. A method for disinfecting a space comprising:
sensing the presence of at least one of bacteria, viruses, dirt, and grime on a surface of the space; and
at least one of selectively activating, projecting, and steering a germicidal light emission from a light source, based at least in part on the sensed presence of at least one of bacteria, viruses, dirt, and grime.

16. The method for disinfecting a space of claim 15 further comprising applying a fluorescent dye.

17. The method for disinfecting a space of claim 16 further comprising illuminating the surface with an excitation emission.

18. The method for disinfecting a space of claim 17, further comprising determining if the space is occupied.

19. The method for disinfecting a space of claim 18, further comprising stopping a disinfection cycle if the space becomes occupied.

20. The method for disinfecting a space of claim 15 wherein the germicidal light is activated, projected, or steered to a control region of the space.
